(19) United States
(12) Patent Application Publication
(10) Pub. No.: US 2011/0172549 A1
(43) Pub. Date: Jul. 14, 2011

(54) HEART RATE AND AN ELECTROCARDIOGRAM MONITORING SYSTEM CAPABLE OF OPERATING UNDER SWEATY, HIGH MOTION AND UNDER WATER ENVIRONMENTS

(76) Inventor: RAVINDRA WIJESIRIWARDANA, Bentonville, AR (US)

(21) Appl. No.: 13/005,546

(22) Filed: Jan. 13, 2011

Related U.S. Application Data
(60) Provisional application No. 61/294,485, filed on Jan. 13, 2010.

(51) Int. Cl. A61B 5/0402

(52) U.S. Cl. 600/509

(57) ABSTRACT

This innovation describes a heart rate and an electro cardiogram (ECG) monitoring system capable of operating under sweaty, high motion conditions and under water environments. The system is tailor made for wearers physical parameters. The system, is capable of wireless transmission of heart rate, ECG via mobile and land line telecommunication networks.

### CROSS-REFERENCE TO RELATED APPLICATIONS

This application claims the benefit of the USA non-provisional patent application 20090292193 filed 2009 Mar. 11 by the present inventor. This application claims the benefit of provisional patent application Ser. No 61/035,852, filed 2008 Mar. 12 by the present inventor. This application claims the benefit of provisional patent application Ser. No 61/294,485, filed 2010 Jan. 13 by the present inventor.

### FEDERALLY SPONSORED RESEARCH Not Applicable

### SEQUENCE LISTING OR PROGRAM Not Applicable

### BACKGROUND

1. Field

This application relates to bio-potential electrodes and sensors based wearable physiological information monitoring straps and garments.

2. Prior Art

Wearable physiological information systems are made by integrating a physiological sensor into the wearable devices including straps, garments and wrist worn head worn devices. Even though these systems have more than 100 years of history, one common problem affects the performance of all these systems. That is these systems fail to perform under most demanding situations such as when a wearer's body undergoes motion and when the wearer is sweating and swim or dive under water. The non-provisional patent application 20090292193 filed 2009 Mar. 11 by the present inventor discusses the motion artifacts reduction under sweaty and high motion conditions. The current invention is a continuation of this research. Improving the accuracy, reliability and comfort level of the system and making the system to work under water.

The second part of the innovation is the signal conditioning and the transmitter unit design. This unit is specifically designed to operate under water conditions and an innovative powering method and an underwater or ground operation detection method are built into the transmitter to save the power.

It was found that there is an optimum size of the electrode contact area with the skin for the maximum signal to noise ratio under high motion and sweaty conditions. 10 People with different genders ages and physical parameters are asked to run at 8 miles per hour under sweaty conditions and ECG signals were recorded for 15 minutes per person and the Signal power and the noise power is calculated by using the recorded. The experiments were carried out with different strap electrodes dimensional parameters and strap dimensional parameters such as width and thickness of non stretch state.

It was observed that when the electrodes sizes are reduced, the noise level is reduced and the signal to noise ratio is improved until this area approaches the critical area value. Further reduction in area results in reducing the signal level and hence it was observed a reduction in the signal to noise ratio. FIG. 2A shows the signal to noise ratio against the electrode area size.

It was also observed that when the strap width is reduced and same strain is applied to the strap the noise level is reduced and the signal to noise ratio is improved until the strap width approaches the critical strap width value. Under swimming conditions it was observed that when the strap width is reducing the slipping of the strap stopped how every further reducing strap width increased the noise and lower the signal level. It was noticed that this critical strap width value depends on the chest size, body weight and the gender. And FIG. 2B shows the signal to noise ratio against the strap width.

Also the signal to noise ratio changes with the strap thickness and shown in the FIG. 2C. It was observed a reduction in noise and increase of Signal to noise up to a critical value and further reduction of the thickness is observed ineffective. The maximum critical value for the strap thickness is found to be 1.5 mm.

The third part of the innovation deals with the two innovative methods that enable physiological information monitoring electrical device with a wireless transmitter having a rechargeable battery to be used under water.

A relay is used to isolate the internal circuit from the external powering pins a relay circuit will connect the battery to the powering pins and disconnects the internal circuit during powering and connects the battery to the internal circuitry disconnects the battery from the external powering pins. FIG. 3A show the electrical circuit block diagram and FIG. 3B shows the powering connector on the transmitter of the relay circuit. This enables a powering pins need not be insulated during underwater operation.

The fourth part of the invention is the process of commercializing the system and it was observed from the that in order to maximize the accuracy, reliability and the comfortability the system needs to tailor made for each individual and the following innovative processes is proposed for the commercialization of the product. The FIG. 4A shows the processes block diagram.

### DRAWINGS—Figures

FIG. 1A—Wearable strap based underwater operable strap system.

FIG. 2A—Graph shows the signal to noise ratio against the electrode area size

FIG. 2B—Graph shows the signal to noise ratio against the strap width

FIG. 2C—Graph the signal to noise ratio changes with the strap thickness

FIG. 3A—Shows the electrical circuit block diagram of the Relay circuit.

FIG. 3B—Shows the powering connector on the transmitter of the relay circuit.

FIG. 3C—Commercializing & ordering processes block diagram.

### DRAWINGS—Reference Numerals

001—Elastic Strap
002—Strap connector arrangement double loop
003—Female part buckles part Strap connector arrangement
004—Male buckle part Strap connector arrangement
005—Electrode connector wire to the transmitter electronics
006—Transmitter
007—Electrode with the ring embodiment patent application 20090292193 filed 2009 Mar. 11 by the present inventor.
008—Female connector of battery charger adaptor
009—Strap width
010—Strap thickness

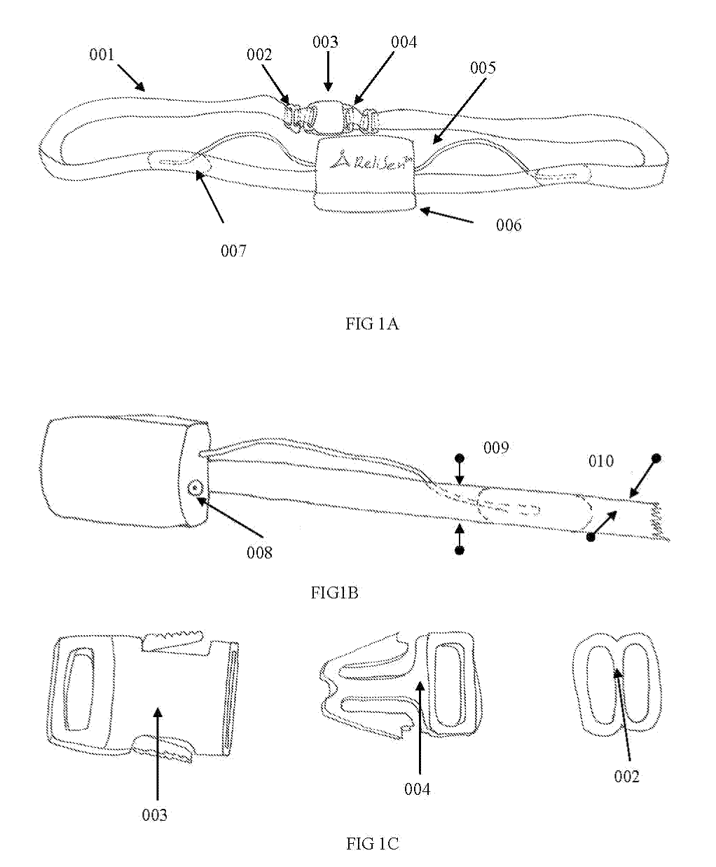

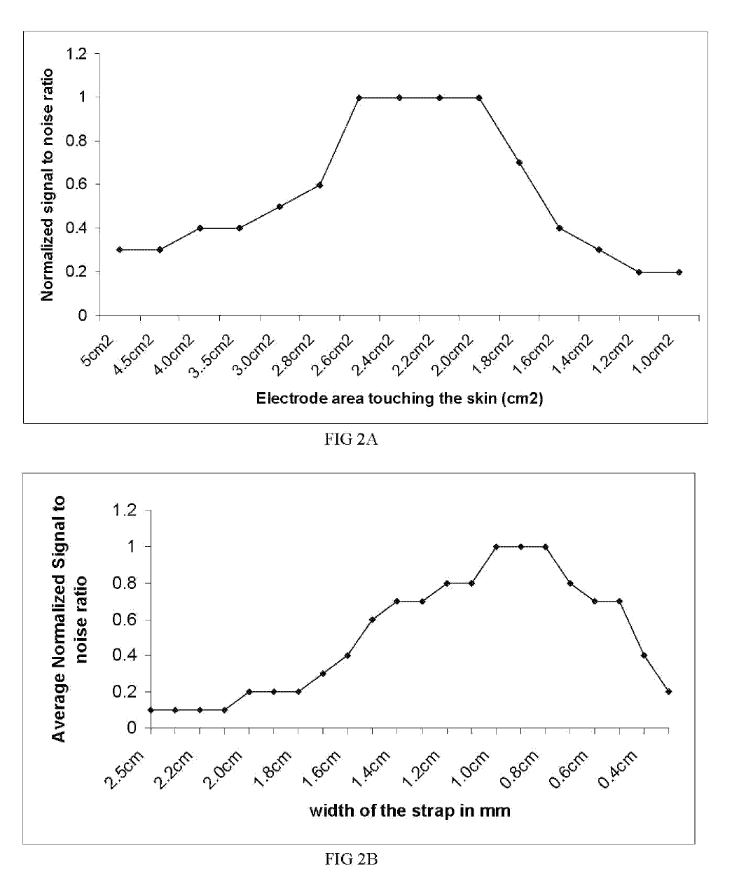

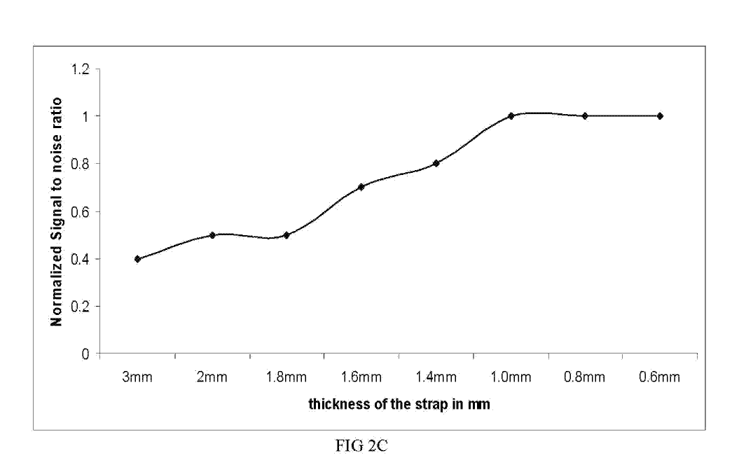

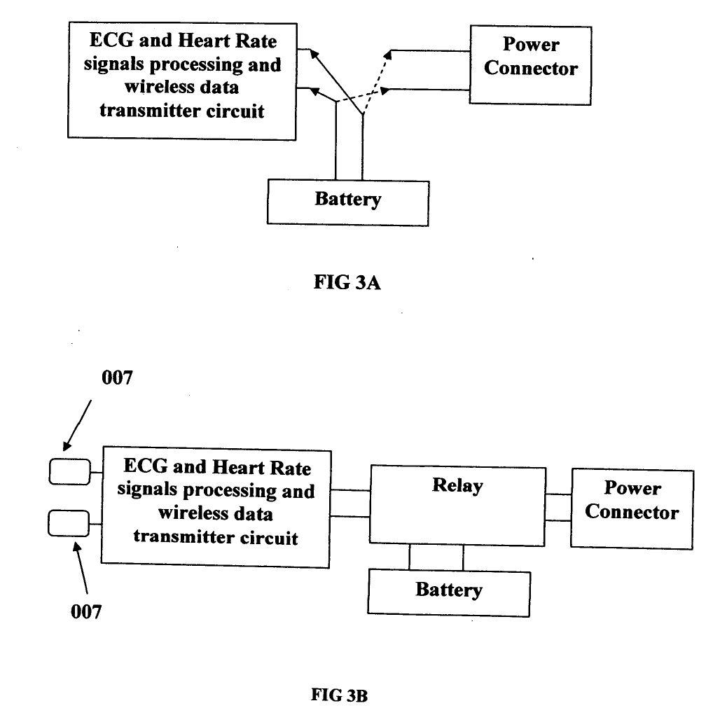

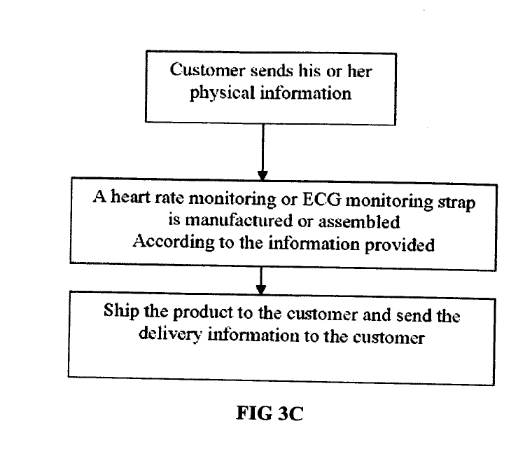

### DETAILED DESCRIPTION OF FIG. 1A, FIG. 1B, FIG. 2A, FIG. 2B, FIG. 2C

FIG. 1A—Shows the stretchable strap (001), transmitter (006), electrodes (007), connector wires (005) strap detachable connector arrangement (002,003,004). The electrodes are attached or embedded in the strap as described in the patent application 20090292193. Upon wearing the system on a person's chest the heart rate information or the ECG information is transmitted to an external monitoring station wirelessly. The ECG signal is picked up by the two electrodes of the strap.

FIG. 1B—Shows the powering pins of the female powering connector (008) of the housing.

FIG. 1C—Shows the connector buckle parts detach and attaché the strap.

I claim:

1. An underwater operable physiological signal monitoring device having an electrical relay device in the powering circuitry covered by a water tight casing.

2. A physiological signal measuring device constructed with a stretchable strap, bio-potential sensors for monitoring electro cardiogram or heart rate such that:
(a) the sensor skin surface contact area is less than 5 square centimeters and greater than 1 square centimeter; and/or
(b) the minimum strap width of any part of the strap is less than 1.5 cm and greater than 0.5 cm; and/or
(c) the strap thickness of any part of the strap is less than 1.5 mm; and/or
(d) the strap is transparent or translucent to light.

3. A physiological information monitoring device according to claim 1 and claim 2.

4. A physiological signal monitoring device according to claim 1 or claim 2 or claim 3 tailored upon receiving the user physical information.

5. A device according to claim 1 or claim 2 or claim 3 where the materials used in the embodiment are being foam, rubber, plastic, polymeric, ceramic and any combination of these materials.

6. A device according to claim 1 or claim 2 or claim 3 where the sensor is attached or embedded or integrated together with the stretchable material and then the embodiment is put in either by using molding or layer construction methods or combination of both approaches.

7. A device according, to claim 1 or claim 2 or claim 3 where the device can be made by using flexible materials such as an elastomeric polymeric materials and a stiffener is used to get the required stiffness and mechanical properties of the embodiment.

8. The sensor surface that touches the skin according to a device in claim 1 or claim 2 or claim 3 having an electro conductive or thermal conductive adhesive layer.

10. A wearable device according to claim 1 or claim 2 or claim 3 having two electrodes switch in any part of the device such that, upon contact with electrically conductive liquid will make a circuit.

11. A device according to claim 4 where the parameters used for tailoring are user physical parameters.

12. A device according to claim 11 where the physical parameters are acquired via internet web ordering page.

13. A device according to claim 12 having an additional reparation monitor or blood pressure monitor or EEG monitor or EMG monitor or blood glucose monitor or any combination of.

14. A device according to claim 1 or claim 2 or claim 3 having wireless transmitter unit where the information can be transmitted to an external base station.

15. A system with multiple devices according to claim 1 or claim 2 or claim 3 or claim 13 or claim 14 where a group activity can be monitored.

16. A device according to claim 1 or claim 2 or claim 3 or claim 13 or claim 14 where the transmission is done via wireless or land lines of public switching telephone network or public switching data network.

17. A wearable device according to claim 10 where the switching action is used to change the electrical parameters such as voltage, power and current for optimum operation of the system.

18. A wearable according to claim 1 or claim 2 or claim 3 or claim 10 having data storage capability.
